(19) United States
(12) Patent Application Publication
(10) Pub. No.: US 2015/0119759 A1
(43) Pub. Date: Apr. 30, 2015

(54) Impact Sensing Mouth Guard and Method

(71) Applicant: Merrigon, LLC, Gilbert, AZ (US)

(72) Inventors: Anthony M. Gonzales, Oak Park, CA (US); Robert Merriman, Gilbert, AZ (US)

(73) Assignee: Merrigon, LLC, Gilbert, AZ (US)

(21) Appl. No.: 14/063,354

(22) Filed: Oct. 25, 2013

(51) Int. Cl. A61B 5/00; A63B 71/08; A61B 5/11

(52) U.S. Cl. CPC A61B 5/682; A61B 5/1122; A61B 5/7282; A61B 5/7445; A61B 5/7275; A63B 71/085
USPC 600/595

(57) ABSTRACT

A mouth guard senses concussive forces, calculates risk factors for head injury, and displays status of risk and potential injury. The mouth guard may be used to identify, treat and prevent exacerbating injury. The mouth guard can be programmed with biometric data to better calculate and anticipate impact thresholds and more precisely predict and prevent injury.

### BACKGROUND OF THE INVENTION

1. Field of the Invention

This invention relates generally to wearable detection and alert electronics that double as protective gear for the prevention of injurious concussive forces. More particularly, this invention relates to a sports mouth guard with built-in on-board electronics for sensing lateral and rotational forces, transforming such data, and communicating estimated risk levels.

2. Description of Related Art

At all levels, athletics are seen as constructive methods of exercise. Sports encourage robust competition and health. Men and women, boys and girls participate in a variety of sports and athletic activities on levels ranging from the personal to informal pick-up game, to the more organized and professional levels. Given the variety of individuals involved, there is a diverse number and range of sports that we play. Some of these games involve high speed running. More physical sports may even involve purposeful or incidental contact between players and/or fixed objects. Such contact raises the potential for harm, including head and brain injury. While football is seen as the primary cause of concussions and long-term brain injury, it is less known that players in other sports also experience a high-risk for head injury and brain trauma. The incidence of concussions in girls' soccer is second only to football, and the combined incidence of concussions for boys' and girls' soccer nearly matches that of football.

Virtually any forceful impact to the head or body involves some risk level for brain trauma. Head injury may occur from collision with another player, an object, or even from a fall. Impact and rotational forces to the head are the leading causes for injury. Brain injury manifests as either neural, or most often, vascular injury within the head.

It is also widely known that the risk and severity of brain injury is related to the frequency and severity of repeated head trauma. A first blow to the head may modify the risk factors for future injury. For instance, a first incidental hit may lower the threshold for injury by a later fall to the ground. Repeated blows and impacts have a greater impact on the risk of head trauma. Even a minor blow, below the normal threshold for injury, may cause catastrophic brain injury if it follows an earlier risk-elevating first impact. Furthermore, biometric data (i.e. gender, age, height, weight, etc.) provide a separate method to determine impact threshold for predicting brain injury.

During play, head injury may manifest as a temporary impairment or loss of brain function; more severe concussions may cause a variety of physical, cognitive, and emotional symptoms. Unfortunately, some injuries cause no immediate or obvious observable symptoms, while even minor symptoms may be overlooked during the excitement of a game. The unknown consequences of prior impacts further exacerbates the risks, by failing to diagnose an injury and take corrective action.

Given the high-risk of injury in all sports and activities, from team sports to personal fitness programs, prior art solutions have not provided a solution that is flexible and precise enough for use in a myriad of routines. For instance, given the extent of electronics and monitoring systems required for head injury assessment tools, products to be worn by players often involve a skull cap or complete helmet. A helmet, while welcomed in permissive contact sports such as football, hockey and motocross, might be out-of-place for tennis, interfere with play for a sport such as soccer, and even presents an added danger on the rugby pitch.

Other products include multiple part pieces that are deployed on the player and can be cumbersome and/or complicated to employ. Additionally, other products do not provide a simple customizable single-piece portable solution.

It is therefore an object of the present invention to provide a single-piece mouth guard for identifying the risk factor for traumatic head injury.

It is a further object of the present invention to provide a diagnostic device that can indicate the potential for injury.

It is another object of the present invention to provide a personalizable risk detector that can reflect the risk factors based upon impact thresholds of a unique user. It is yet another object of the present invention to provide an instrumented mouth guard that can be widely deployed to assess and indicate injury risk.

All these and other objects of the present invention will be understood through the detailed description of the invention below.

### SUMMARY OF THE INVENTION

The present invention is a mouth guard for detecting, measuring and indicating impacts and calculating the magnitude thereof combining such impact data with preprogrammed user biometric data to display risk factor. The mouth guard includes a mouthpiece to be worn by the user while participating in an activity or situation in which there may be sudden force or movement that may lead to concussive head trauma. The mouthpiece is powered by a power source, which may be a battery, preferably one embedded in the mouthpiece for powering the on-board electronics. The mouth guard also includes an accelerometer, for measuring linear forces, and a gyroscope, for measuring rotational forces, preferably within the mouthpiece. The power source powers a processing unit in the mouth piece that receives data from the sensors. The processing unit may conduct a calculation to determine if a significant impact has occurred. The processing unit may have hard-coded levels for predetermining impact thresholds, or may be programmed by an individual user for a personal threshold scheme. When the sensors detect an actionable impact force, the processing unit caused a display indicator, preferably a light source, preferably on the mouthpiece, to display a light to indicator to the user and others around that a significant force has been sustained. This light display may warn of potential future head trauma, current head trauma, and indicate for the user to cease the activity and seek a safer venue free from a high risk of future sudden forces.

The display indicator may be a light, preferably a low-power required light, such as a light-emitting diode (LED) that can display a solid and/or blinking light in one or more colors; preferably the LED is a three-color light, allowing display of a myriad of color wavelengths. The display indicator may be embedded in the mouthpiece, preferably at the front or lower front, or may be attached or extending outwardly in front of the mouth guard. The display indicator may use a single color to show that it is on and functioning to detect impacts, and another color or lighting scheme once an actionable force is detected. Blinking and solid lights of various colors may be employed. For instance, the system may use a first color, such as blue, and blink to indicate a minor impact has occurred. A second minor impact may show a solid blue light. At any time, a major impact may trigger a red light display. Other displays may be available.

The calculation of impact thresholds may be dependent on personal or universal data. For instance, a personalized mouth guard for specific aged person of a certain gender may set thresholds specific to that person. Preferably, there will be various categories for each of these biometrics, including a range of weight, years, etc. The mouth guard may record past hits and use these in calculating or altering the threshold(s). For instance, one minor impact may lower the threshold for the next minor impact.

Preferably, the mouth guard will be able to sense an array of forces, and include multiple impact force thresholds. For instance, the mouth guard can distinguish between a major force and a minor force. In addition, information of past recent impacts may open new thresholds, or lower the threshold for minor or major impacts. The processing unit may have an on-board memory for temporarily or permanently recording past impacts. The processing unit memory may be reset either at a given time period, i.e. recording data for 24 hours, and/or may allow for a reset button to erase some or all past impact data.

Furthermore, the mouth guard may include an on-board data input system, such as a button key for input of various factors, such as biometrics of age, weight, height, and gender. The biometric data may set the impact thresholds for future use. The mouth guard may come equipped with an ON button that may also perform a shut down or send the mouth guard to a low-power stand-by mode. The processing unit memory may be power dependent, such as typical random-access memory (RAM) or may include hard coded memory that will persist after the mouth guard is shut off or the power source fails, such as flash memory. To avoid power failure, the power source may be on-board and be rechargeable such as through an inductive power recharge.

The present invention also includes various methods for detection, calculation and display of potential head trauma, preferably by use of a mouth guard with on-board sensors and diagnostic logic. The mouth guard is worn by the user and placed alongside the jaw or preferably molar teeth within the mouth. The user can preset a threshold scheme by putting or selecting a preferable predetermined biometric profile, preferably via on-board input button. The preset can be used to focus the diagnostic logic on particular thresholds of rotational and/or linear forces.

The may include on/off switch to conserve battery. Preferably off position option will allow any required memory to store attributes, such as biometrics, or previous shock instances. On/off switch may include an On button when pressed a first time to activate mouth guard until battery death, and a standby mode to allow low power mode between uses. Mouth guard in stand-by mode, preferably includes accelerometer function to allow automatic on-switching when sensing a major impact, or just even a minor motion indicating future use.

While powered on, the sensors continually monitor forces. Once the sensor experiences a force beyond the preset threshold, the logic function communicates with the mouth guard to cause the display function to illuminate in a predetermined scheme, such as a lighting scheme, preferable for an on-board LED lights.

### DESCRIPTION OF THE DRAWINGS

A more complete understanding of the present invention may be derived by referring to the detailed description when considered in connection with the following illustrative figures.

FIG. 1 depicts a perspective view of an embodiment of the present invention;

FIG. 2 depicts a top view of an embodiment of the present invention;

FIG. 3 depicts a perspective view of an embodiment of the present invention;

FIG. 4 depicts a top perspective view of an embodiment of the present invention;

FIG. 4A depicts a top view of an embodiment of the present invention;

FIG. 5 depicts a side view of an embodiment of the present invention;

FIG. 6 depicts a linear/rotational force impact scale indicating risk factors based on a combination of forces;

FIG. 7 depicts a side view of a human user head;

FIG. 8 depicts a top view of a human user head;

FIG. 9 depicts a top view of an embodiment of the present invention;

FIG. 10 depicts a side view of a human user head engaging an embodiment of the present invention.

FIG. 11 depicts a linear/rotational force impact scale indicating risk factors based on a combination of forces;

FIG. 12 depicts an alternative linear/rotational force impact scale indicating risk factors based on a combination of forces;

FIG. 13 depicts an alternative linear/rotational force impact scale indicating risk factors based on a combination of forces;

FIG. 14 depicts an alternative linear/rotational force impact scale indicating risk factors based on a combination of forces;

FIG. 15 depicts an exploded view of an alternate embodiment of the present invention;

FIG. 16 depicts a preferred gyroscope with directions of detectable angular rate; and

FIG. 17 depicts a preferred accelerometer with the direction of detectable accelerations.

### DETAILED DESCRIPTION OF THE PREFERRED EMBODIMENTS

The present invention may be further described and understood by a limited set of preferred embodiments. However, the embodiments described herein are intended for illustrative purposes only, and not to preclude other devices or embodiments that embody the invention herein.

A preferred embodiment uses a mouth guard to be placed into a user's mouth. The mouth guard is self-sufficient as a small portable useful item that can be used in a myriad of occasions and activities. It is anticipated that the user will be engaged in some sort of athletic activity. The mouth guard will be equipped with electronics that allow monitoring or sensing of forces, both linear and rotational. Preset thresholds, preferably personalized for the particular user, of combined rotational and linear forces will indicate various risk factors for brain injury. Furthermore, certain repeated hits of various or same intensity will indicate risk. Certain hit, or hits, may modify the risk threshold for future hits in the near term. When operating, the mouth guard may indicate status “ON” by lighting up an on-board display. Should a minor impact occur, the display may indicate so, for instance by blinking light, or light of another color. Should a major impact occur, the display would indicate, for instance in an alternative lighting scheme, i.e. color. The thresholds may be preset, and on-board memory may recall prior recent incidents to modify impact thresholds without further manual intervention.

A preferred embodiment of the present invention includes a mouth guard with on-board electronics and signaling, sensors and display lights. An LED indicates status of the system including powered status and risk factors. The use of on-board display alleviates the necessity for a third party device or extra component as is necessary in many competitive products on the market.

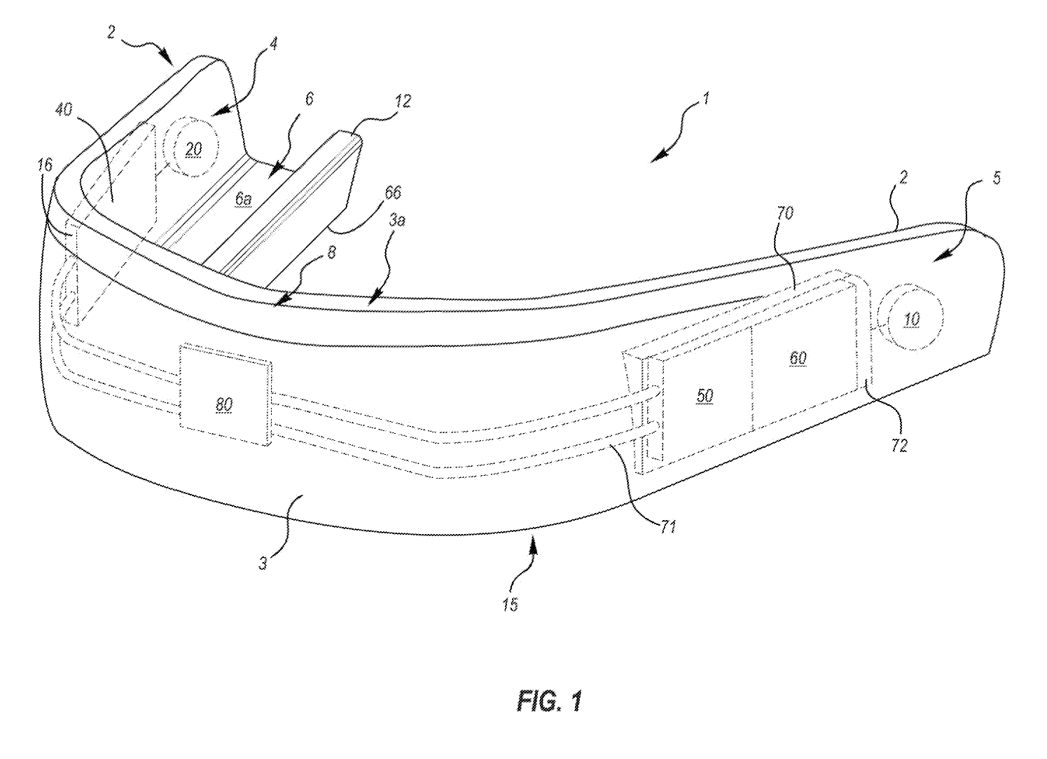

A first preferred embodiment of the present invention is presented in FIG. 1. Mouth guard 1 includes the usual components of many safety and impact reduction mouth guards known in the art. Body 15 is formed with a substance that preferably a soft rubber or plastic and acts as a resistor to electric current. Common materials include thermoplastics that melt and deform at a certain high-temperature to allow for an initialization in hot water bath of −180 degrees F for about 30-60 seconds. Once in a more liquid and less viscous state, a user can customize the mouth guard by deforming the body to fit the actual contours of a user's teeth and mouth (not shown). In the preferred embodiment, the mouth guard will be customized for one user's fit.

Body 15 encapsulates flex board 70 and all components thereon. Flex board 70 may be limited to certain components, flex board may come in multiple pieces, each piece carrying one or more component, or all electronic may be including on a single flexible circuit board, preferably encapsulated within the mouth guard. Microcontroller 16 provides on-board preprogrammed logic to collect data from sensors, preferably including gyroscope 60 and accelerometer 50, transmitted along embedded wires 71. It is preferable that microcontroller also contains preprogrammed thresholds, and various sets of impact threshold data. The sensors, accelerometer and gyroscope, may alternatively be positioned intermediately within the body, or padding element, or at an interface between a hard section connected to such body. Alternatively, electronic components may be connected by wire, electronics pods, etc. instead of a flexible circuit board.

Mouth guard may be formed in three basic steps. First, the flex-board and all components are arranged. Once arranged, the electronics may be set within a bottom mold. The bottom mold is then filled to complete the lower portion of the mouth guard with a material to form the body. Finally, an upper chamber mold may be used (preferably once the lower portion is flipped) to complete the body portion. Once completed, the body is then cleaned, and the action/power buttons identified and ensured for indentation. Alternatively, the body may be formed in a single injection mold around the electronics, or any other method as known in the art. The electronics may be protected by a casing or shield to avoid overheating during production encapsulation. The casing may adhere to, or otherwise be absorbed into the body to form a tight fit between the electronics and body. The electronics, particularly sensors, must adhere strictly to the body to allow for precise measurements of force on the body. Therefore, outside surface of the sensors may be exceptionally thick to expose to the body during formation/injection, or the surface may be scored, pocked, or otherwise detailed to allow for snug tight fit with body.

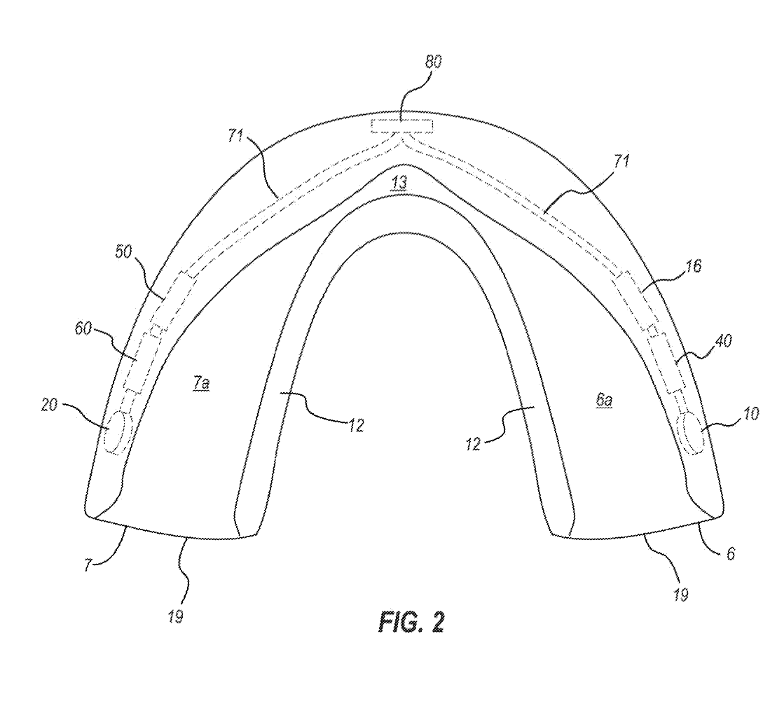

Referring to FIGS. 1-2, mouth guard 1 includes a right bite pad 6 and a left bite pad 7 to contact user's teeth, preferably molars and/or bicuspids. Bite pads 6 and 7 may be partially deformed in the initialization procedure to better conform to the contours of a user's mouth. Bite pads and side shields will be thick enough to allow such deformation without exposing electronics. A channel may be formed to receive the teeth. It is also contemplated that bite pads may include at least two substances, a first at bite pad top 6A and 7A, and a second material such as a hard plastic or shield at bite pad bottom 6B and 7B (seen in FIGS. 1 and 5). Front bite pad 13 is similarly formed to accommodate incisors and cuspids.

Side shields 4 and 5 may provide housing for on-board electronics and may also be made of deformable plastic or rubber, or other material or composite. Alternatively, side shields will be made of a more permanent solid material for the protection of the electronics, and may or may not be coated with the deformable material for better user sensory feel along users inside cheeks. Interior top ridge 12 of front shield 3 includes protection and front shield 3 provides for encapsulation of display 80. Display 80 may be set right along edge 3A or more centered within front shield 3. Front shield 3 preferably includes indent 8 at the top to accommodate the superior labial frenulum. Interior top ridge 12 should be soft enough to accommodate contact with user's soft gum or gingiva. In this embodiment, the bulk of the electronics are positioned on the sides and front of the mouth guard to avoid the risk of a hard bite to damage components. Alternatively, the electronics can be placed in the front, or below with a more solid bite pad, or in combination of the two, or elsewhere.

Microcontroller 16 preferably collects information from sensors, performs necessary calculation, and when impact data received from sensors indicates a hit beyond a threshold, microcontroller 16 sends signal along wire 71 to display 80 to cause display 80 to demonstrate a predetermined indication scheme. Most preferably, the data is passed along in real time, although in some versions the data is stored in a memory and accessed at a later time. Memory is preferably stored in or accessed by the microcontroller, but may also be included in a separate element (not shown) such as RAM chip(s), flash memory, etc.

Preferably display 80 includes light-emitting diode display. Action button 10 may serve as an on/off toggle switch for the mouth guard electronics. When in the off-position, the mouth guard should still serve the standard purpose of a simple mouth guard, but not collect or handle impact data. Input button 20 allows user to communicate and send direct data in predetermined signal language to the microcontroller to accomplish certain tasks such as setting a predetermined biometric set of thresholds, reset the device, reset the timer, or in some embodiments, set the device to standby mode.

Various demographic modes are contemplated for customizable programming of risk factors/thresholds. For instance, there may be a high, low, and average threshold category for weight. There may also, or instead, be a gender category, and/or an age category, etc. In one embodiment, to program the mouth guard, press the action button for 10 seconds. The indicator will blink white to indicate that programming is now available. Press the action button once for low mode, twice for medium/average mode, and three times for high threshold mode (weight). Each time the button press is activated, the display may change colors, i.e. turn red briefly to indicate acceptance of a button press. Holding the button an additional at least 10 seconds, will set into a second mode to indicate gender, i.e. once for male, twice for female. When inputting a second demographic data set, it is contemplated that a second color will blink in display, i.e. blue blinking. When inputting the third demographic set, i.e. age, the same rules will apply for age categories, i.e. under 12, 13-16, 17-22, 23-35, and 35+. The mouth guard is sold with an instruction manual to allow for activation and customization, as well as instructions to reset the customizable category. Depending on the customized demographic data of a user, the impact thresholds will be set. In the embodiment with RAM memory requiring power to retain information, it is contemplated that a low power source will maintain demographic data in standby mode. Impact thresholds will be set to raise or lower thresholds for minor and major hits, and/or modify the risk curves based on gender or age data, for instance see curve modification indicated in FIG. 12.

Further, preferably on flex board, is power source 40, preferably a battery. Power source 40 preferably provides power as direct current to microcontroller 16, display 80, and preferably sensors 60 and 70. Battery voltage may be below 10V and preferably between 1V and 6V. A voltage regulator (not shown) may be included to allow a single power source to provide power for all components.

Power source 40 may be a simple coin cell battery. Alternatively, power source 40 uses inductive or wireless charging. Inductive charging allows a rechargeable guard while still not having any exposed ports. In a preferred embodiment, there may be a complementary charging station, i.e. pad or mouth guard container case, with built in inductive capability.

In an alternative embodiment, antenna 71 may be included along flex board 70 to allow for remote transmission from or to on-board electronics. For instance, remote data or instructions may be programmed to the mouth guard from a remote component, such as over wireless frequency Wi-Fi, or other electro-magnetic transmission, to communicate data to the on-board micro-controller. Another use may be as a source of information to communicate impact data and risk factors to an off-board monitor.

Microcontroller 16 preferably includes built-in memory capacity. Preferably, a portion of the data in memory will be hard coded. Preferably the predetermined biometric scales and impact threshold, as well as the logic equations for one or all of the biometric sets will be hard-coded into memory. The memory may be able to hold, and selectively erase, historical impact data. It is envisioned that through, input button, the memory may be reset to erase short term memory of historical impacts. It is also contemplated that the memory may have on-board clock timer that will be used by microcontroller to selectively erase historical impact data more than a predetermined time length, i.e. more than 24 hours, while using more recent data to help determine if an impact threshold has been met.

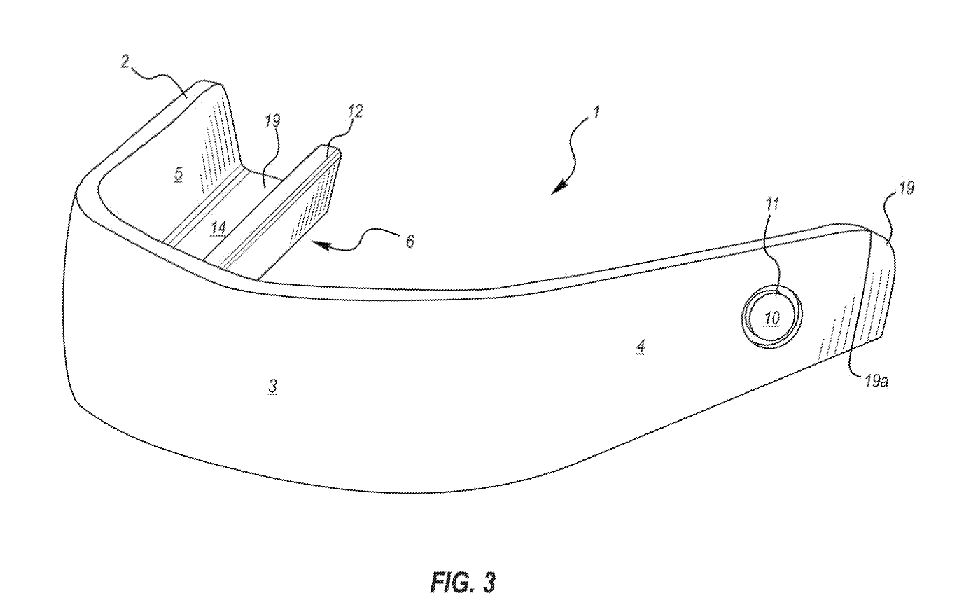

Embodiment shown in FIG. 3 shows the user impression of mouth guard 1. Action button is shown on the outer side of the mouth guard and recessed within button indention 11 on the circumference of the button to preserve the feel of an ordinary mouth guard without any unnecessary user discomfort. Excess molar portion 19 may be included to allow users with a smaller mouth cavity, or preferring a smaller mouth guard to disregard, remove and eliminate excess portion. Components may be arranged to allow for a variety of user sizes, with sensors still in contact or coupled to proper bicuspid or molar teeth to allow for accurate measurement of forces. Indication bar 19A may show user the limit or portion to remove without affecting on-board component otherwise hidden within guard.

As seen in FIG. 17, an accelerometer serves as a linear forces sensor. Preferably includes sensors for three dimensions. Preferably, the sensor fits within the mouth guard connected by wires to the microcontroller and other components. A small thin accelerometer such as the a 3×3×1 mm³ motion sensor with a digital output, low power requirement, high-g, and 3-axis accelerometer, as known in the art, is preferred to suffice. It is preferred that supply to the accelerometer is below 2V, around 1.5-1.8V, but may be as high as 3.6-6V. It is contemplated that in a low-power mode, the accelerometer can suffice on as little as 10 micro amps The accelerometer may be able sense forces as low as 10 G, and to handle forces as high as 100-400 G, and have a high shock tolerance above 1000 G. A sleep or stand-by mode may be used to conserve power. The accelerometer may have on-board logic and memory to log impacts, or to selectively report only impacts above a certain threshold, to save power. It is contemplated that the accelerometer will transmit digital signals.

Thresholds for linear forces for an average adult male may be set as high as 100 to 300 G forces. Preferably the sensor is able to handle and distinguish forces at this great shock within a 10-20 G range. Lower shocks with impact G force of less than 100 will preferably be selected within 5 G. While the thresholds are listed in this specification and on the Figures, they are in no way intended to limit the threshold settings ranges for practice of the present invention. As studies, data, and even personal preferences evolve, various threshold levels of acceleration and rotation may be programmed into an embodiment of the present invention.

As seen in FIG. 16, the gyroscope should have similar electronic, power, output, and sensor characteristics as the accelerometer. The gyroscope will sense three-axis rotational acceleration, typically considered in radians (rads) per second squared (sec²). Similar in size and shape to the preferably with the linear sensor/accelerometer, the gyroscope will also preferably fit on the flex board The gyroscope either outputs information on the three-dimensional level, or includes in component logic to output a single data packet to include a total G rotational force profile. Given the fixed orientation of the gyroscope in the mouth guard and known placement in the mouth, three-dimensional forces may help determine the location (source) and direction sensed by an accelerometer can on its own or in combination with gyroscope help calculate force source and risk potential. Typically, in forceful impacts, rotation can reach accelerations on the order of thousands of rad/sec², and the gyroscope will preferably be able to determine rotation to the 10 rad/sec precision. In some embodiments, the gyroscope will include on-board memory as the accelerometer, and may also include temperature sensor that may be used to help calibrate impact data. It is contemplated that the gyroscope and accelerometer will perform under the same specifications and actions.

As seen in FIG. 1, it is contemplated that the position and orientation of the gyroscope be fixed within mouth guard 1, possibly held in place by flex board 70, wires 71, and/or relation to other components. This positioning may help determine the source or direction of impact. Data from the gyroscope, accelerometer, or a combination of data between the two (possibly calculated within the microcontroller) can help determine the direction of impact to the head or body.

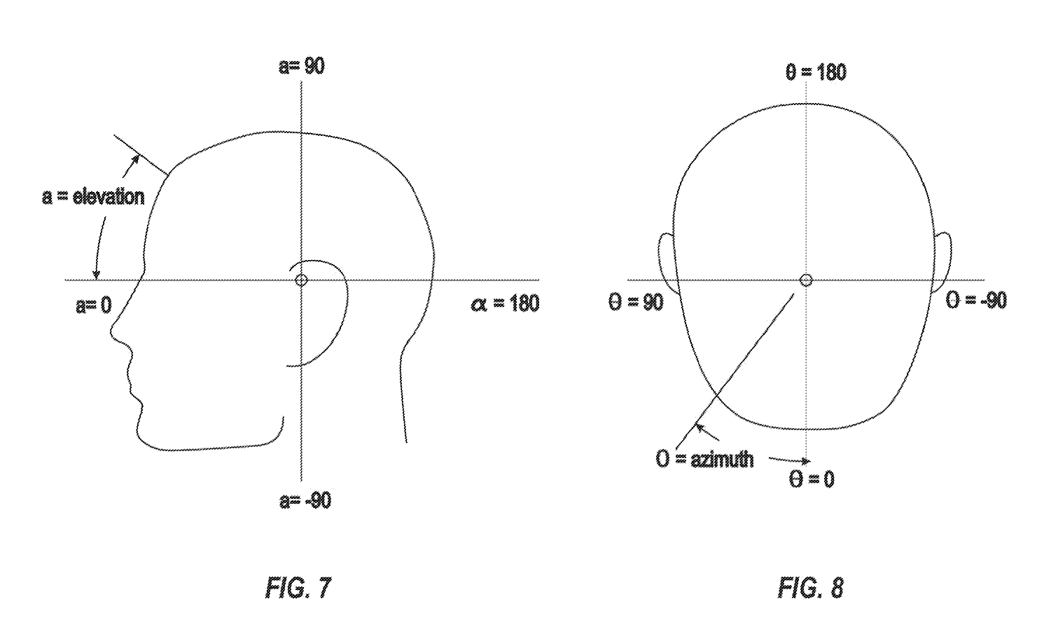

As seen in FIGS. 7 and 8, direction of impact may have an impact on the threshold for risk. For instance, hits in front (experienced as directly back and down) may prove having a lower risk of injury at a high impact force, as forces to the side (experienced as a sharp turn and/or sidal force) may raise the risk (and thus lower the threshold) for risk. FIGS. 7 and 8 indicate a map of the human user head which can help provide illustration for categories of direction hit. Impacts with an elevation greater than 65 degrees may be categorized as a hit to the top, whereas those below 65 degrees, and having an azimuth between −45 and 45 degrees would be categorized as impacts to the front. Elevation below 65 degrees and azimuth between −135 and 135 degrees might be categorized as hits to the rear. The remaining impacts might be categorized as shocks to the side of the head, given the symmetry of the human head about the sagittal plane.

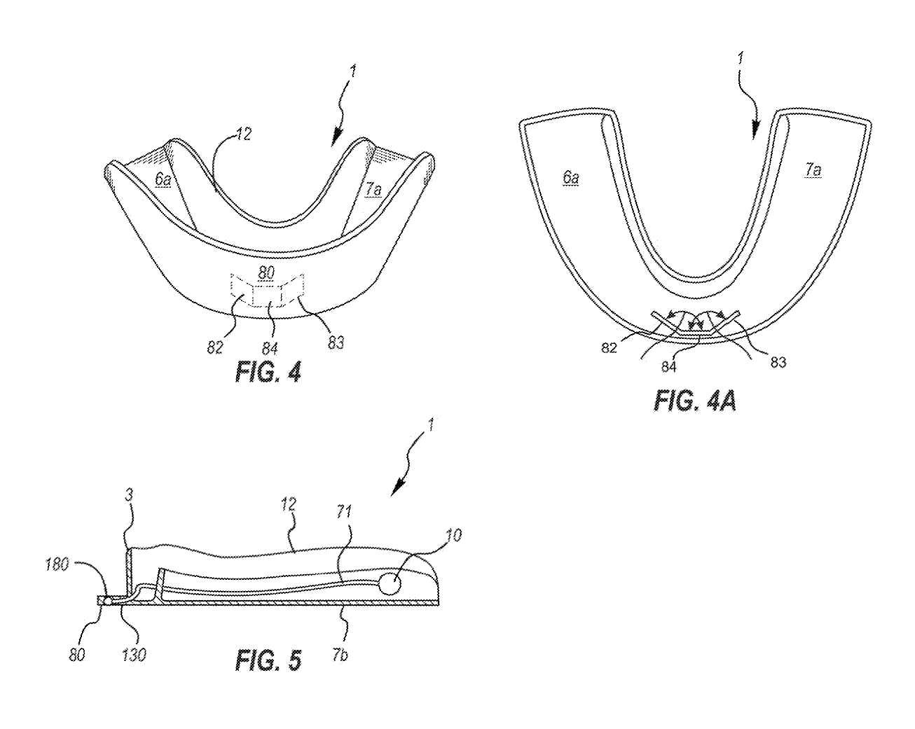

Preferably a single 3-color LED capable of RGB colors, including ability to combine to provide virtually all colors and white. As seen in FIGS. 4 and 4A, three separate LEDs may be provided. Orientation of LED with 120 degree, may be utilized to provide 180 degree or further display viewing, up to and perhaps more than 300 degree viewing, plus the scattering effect on the skin. LEDs may be powerful enough to be viewed when covered by the lips/cheek as the user's mouth is closed.

An alternative display configuration is shown in FIGS. 4-4A. In this instance a triple angled display screen, preferably including a single LED display on each face, is arranged to provide notification to others at the front and sides of the user. An embodiment of the invention also contemplates that the display indicator may be bright enough, or use vibrations with a separate component, to indicate to a single user when in use in an individualized capacity. The display 80 includes front display panel 84, as well as right and left display panels 82-83. In a preferred embodiment, the arrangement is set where by panels are each offset at an angle of about 120 degrees to provide an array of viewing angles. The display may be encapsulated in the padding material, or may be attached on the outside of the display.

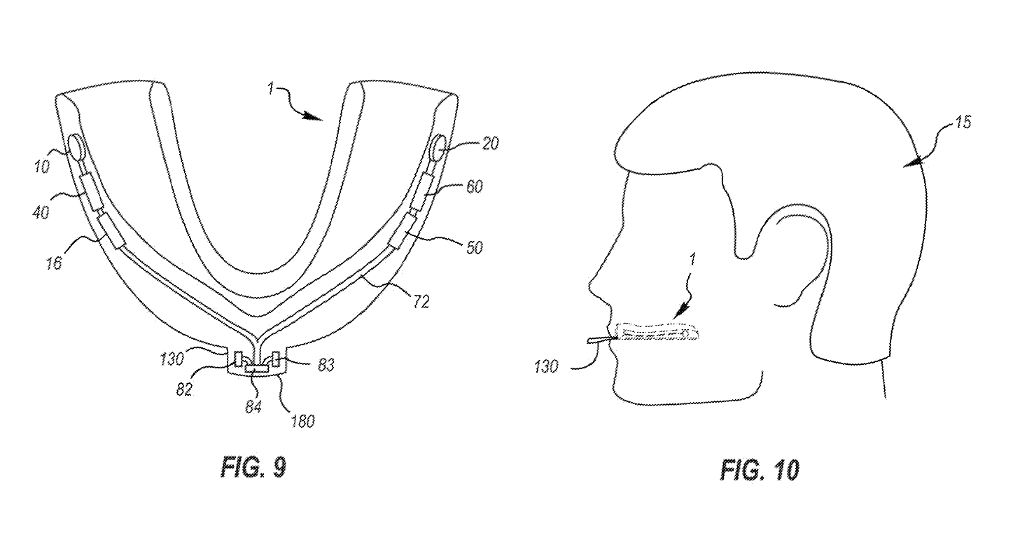

An alternative embodiment is shown in FIGS. 9-10. Mouth guard 1 includes extended tab 130. Extended display 180 is connected to microcontroller, or other component to activate display when necessary. In the embodiment shown in FIG. 9, extended tab 130 includes extended display 180 with three separate LED display panels 84 facing directly forward, and 82 and 83 as right and left displays preferably arranged perpendicular to the front display.

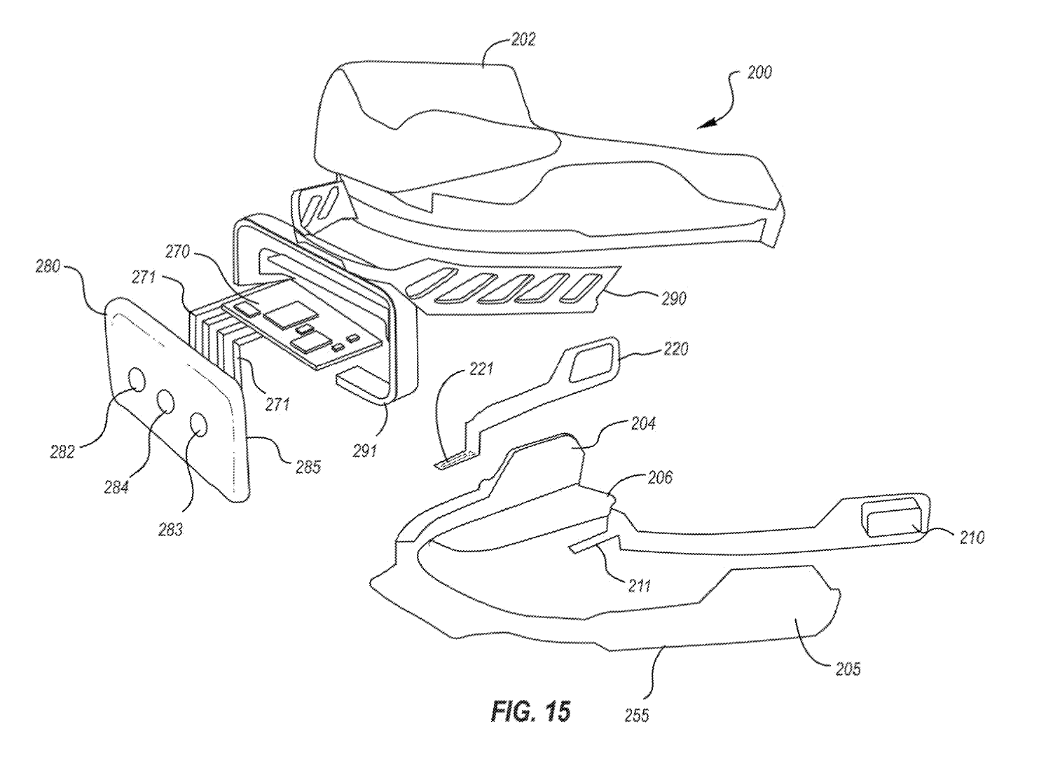

In a preferred embodiment as shown in FIG. 15, three LEDs. A single small board contains all of the components towards the front of the unit all in one location. The present invention may also utilize a screen of multiple LEDs, or alternative light source, to display graphics, colors, or words and/or lettering to communicate risk status. Mouth guard 200 includes a encapsulation material body 202 that holds components. Protection shield 290 serve to provide substance and strength to the body to protect components. Action tabs 220 and 210 serve to allow user input into device. Side shields 204 and 205 may contain conductive material to act as a single antenna 255, or may serve as protection for signals transmitted to flex board 270 from pads 210 and 220 via contacts 211 and 221. Contacts 211 and 221 mate with board 270 which houses the microcontroller and all sensors and power sources, electronic components, etc. Wires 271 communicate with display 280 (off of board) to allow three LEDs 282, 283 and 284 to display status. Frame 291 serves to further protect and hold board 270 in place. In a simple version, each LED may transmit a separate color, in more advanced embodiments, the LEDs each contains multi-color functionality (three lights to create all colors including white), or display panel 285 may contain a myriad of arrayed LEDs or other display lights or indicators to demonstrate various symbols, numbers/words, etc. The display board may also indicate team affiliation (colors) or be used for novelty, i.e. fangs, blood, grass, fake teeth, tongue, cigarette, etc. or be used to display a message or other personalized feature.

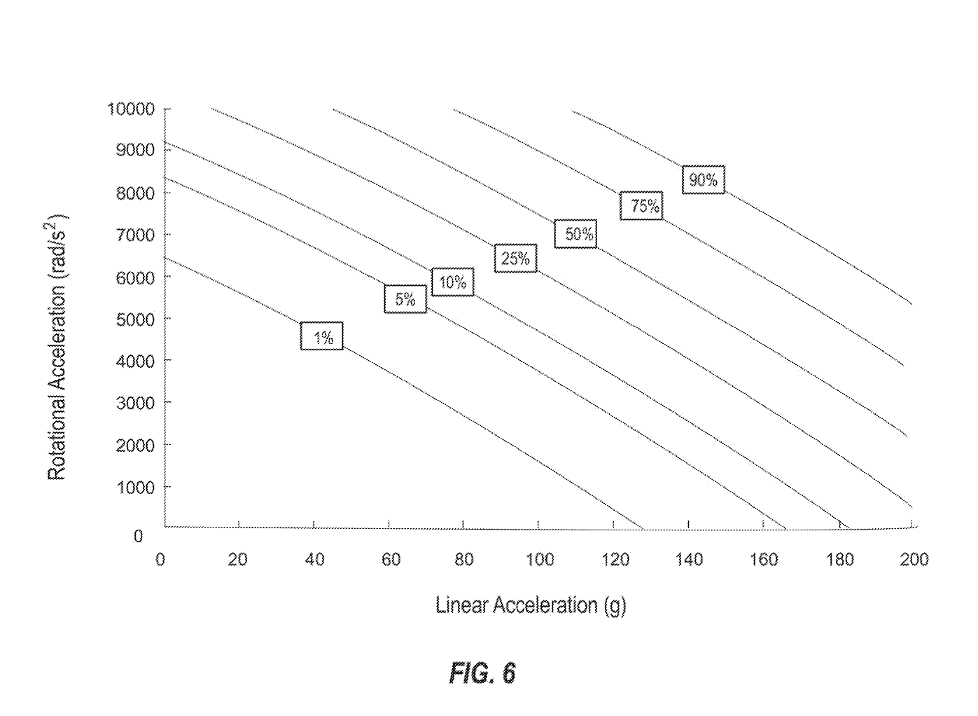

As seen in FIG. 6, risk factors for individuals experiencing shock trauma to the head or body typically manifests as a combination of linear and rotational forces. Even straight shots typically include a partial rotational element given the anatomy of the head and neck. However, straight shots, as well as shocks that have little to no linear component and are merely a rotation will also be included as examples in the logic. Peak rotational velocity may also be measured and combined into the risk algorithms The predictive capability assessment risk function, absent corrections as detailed above and those known in the art, can be displayed as an equation. Where b0 [beta 0], b1, b2, and b3 are regression coefficients determined using a generalized linear model technique, a is peak linear acceleration, a [alpha] is peak rotational acceleration, and CP is the combined probability of concussion.

$$CP = \frac{1}{1 + {e}^{- ( {β}_{0} + {β}_{1} a + {β}_{2} α + {β}_{3} a α )}}$$

While linear acceleration is not a significantly worse predictor of concussion than the combined probability of linear and rotational acceleration for concussion for all data sets, and rotational acceleration alone is associated with the smallest predictability, the purpose is for a predictive capability with a low false-positive issue. Such accurate information with low false-positive indications should lead to greater adoption and continuing use of the product. Using rotational acceleration as a brain injury predictor results in the greatest false positive rate associated with high true positive rates, while using the combined probability of concussion produces lowest false positive rates in all head impact telemetry data sets. Findings clearly indicate the a combination of both linear and rotational forces add value to the safety of the device, particularly among young athletes, who will resist sitting out of a game due to a false positive. The goal is to prevent players staying on the field with a concussion, while simultaneously encouraging product adoption and use.

For illustration, using a “red” display might indicate that the risk threshold is met. Using rotational acceleration measurement leads to more often “going red” and the player not having a concussion, while simultaneously having a more hits that previous would not have “gone red” head only linear acceleration been used, resulting in a concussion. Additionally, the curve may be modified to include threshold of a single source, i.e. accelerometer or gyroscope, as shown in the intersection of the probability lines of FIG. 6 with the axis.

Plotted out, risk function predicts probability of concussive impact. As shown in FIG. 6, for a typical user, the thresholds for various risks are displayed charted as a combination of linear and rotational accelerations (indicating force). In another preferred embodiment, the user may preselect the level of probability for the various risk levels. In a preferred embodiment, a set risk factor, i.e. 50% may be set as a major hit, as recorded by the sensors and calculated by logic at the microcontroller. A signal would be sent to activate the display indicator to show a significant hit, such as “RED” light. A hit in a range below a certain low threshold, i.e. below 25%, but above 10%, might indicate a preparatory hit that reduces the threshold and increases the risk factor for a second force causing a concussion. In this instance, the major hit threshold might be reduced, i.e. from the 50% equation level to a 40%, etc. A minor hit, i.e. between 25%-50% probability of concussive brain injury, might signal a first warning display, i.e. blinking light, and also may modify the thresholds for major and minor hits in the same fashion of a low hit, or otherwise.

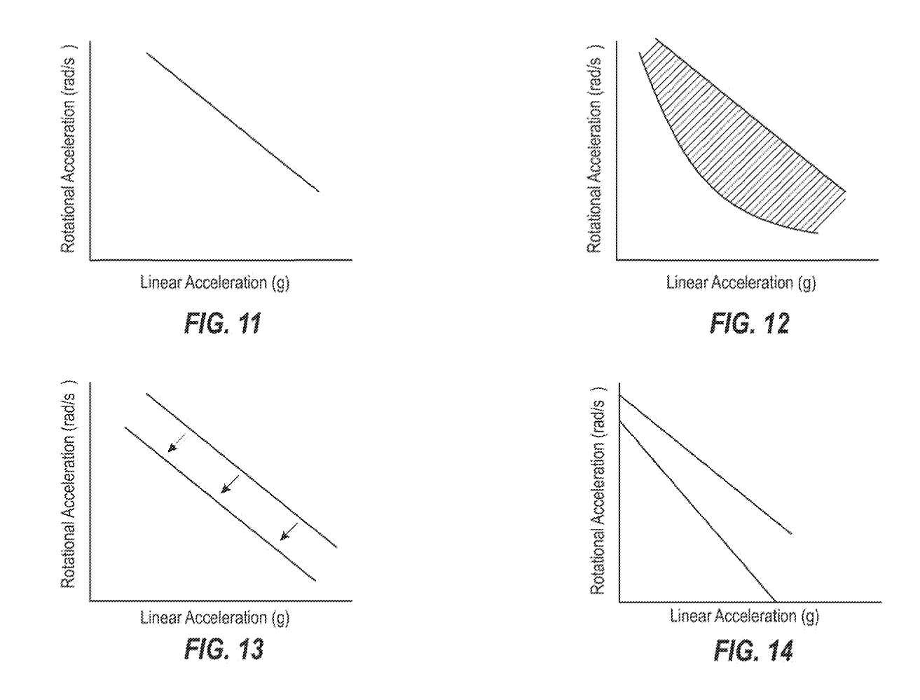

Typical procedure of risk factors, impact thresholds, are demonstrated in FIGS. 11-14. FIG. 11 demonstrates a typical mainline threshold. Upon modification based on biometric data, the threshold may shift simply, as shown in FIG. 13 or dynamically, as in FIG. 12. It is contemplated that certain demographics may be more or less susceptible to injury based on linear vs. rotational forces, i.e. children may be more susceptible to rotational forces, so the modified risk factor/threshold might be lowered disproportionately in the rotational dimension as compared with the linear dimension. A first impact of significant shock may impose a shift of the major risk threshold simply as in FIG. 13, or may also dramatically alter the probability function as shown in FIG. 14 demonstrating that a portion of the brain susceptible to specific type or direction of impact may be at a higher risk.

The mouth guard will preferably be powered by on-board power source, such as a battery. It is compatible with an embodiment with out on/off switch could have on switch such as broken capsule that may be a one-time use as switch. The capsule may contain a resistor that, when broken, serves to transmit electrical power and thereby power on the device. If using RAM for memory, a low-power standby mode may be used to conserve power. Action button, i.e. 10 or 20 shown in FIG. 2 may be used to power down the device, or device may use a clock timer to monitor activity and auto power down after a set period of inactivity, i.e. four hours.

Various LED Display settings are contemplated. For instance, when the device is on and actively sensing forces, the display may show a solid blue light. This will indicate that the device is on and functioning. Before activity ensues, each player may check the status of the device, and players with a non-functioning mouth guard may be identified. A minor hit may cause a different display, i.e. blinking red/green or alternative colors. The differing display may be reset, should the player chose to resume play by either waiting a set amount of time, i.e. 5 minutes, or by resetting via the on-board action button. During play, if there is a first concussive shock that triggers the alternate (lower) thresholds, this may be reset by action button, i.e. holding it down. This may be done when the thresholds are not properly set, to avoid false positives, or to allow multiple users to use the device. When a major hit occurs, a solid red display light might indicate high risk of injury and alert player to be removed from play. Further advances with multiple LED may allow for a more detailed display, i.e. not only using color, but also a letter, symbol, or word, or percentage risk factor, etc. may be displayed. This advanced multi-LED display can also be used effectively when initially setting risk thresholds manually for better interactivity.

It is preferable that the power source include an on-board battery, preferably Nickel-Cadmium as known in the art, to provide necessary voltage power for all components. It is contemplated that in a preferred embodiment, the battery will be built into and integrated encapsulated within the mouth guard. In an alternative embodiment, the on-board battery will be rechargeable. The recharge may be accomplished by a hidden pug in accessory access port, preferably behind a flap section of the mouth guard material (not shown). In another alternative embodiment, the battery can be recharged remotely by induction, preferably through a complementary pad docking station, or alternatively within a mouth guard case.

In an alternative preferred embodiment, there will be a complimentary display function on a remote display piece. In this instance, the on-board, or integrated built-in, antenna transmits a preferably electro-magnetic signal to the remote receiver which is in turn connected to the display. An example might be a remote WiFi receiver, such as a common handheld device, i.e. cellular phone, or WiFi handheld tablet, etc.

The alert and indication are part of the present invention. An LED display is contemplated as a preferred embodiment of the alert method, but future and more advance device could integrate alternative indication, such as: text, email, push-notification, sending data to an external app, and that app then alters the individual risk factors. The present invention has been described in the above illustrative embodiments, but should not be considered to be limited in any way therein.

We claim:

1. A mouth guard for detecting impact and calculating the magnitude thereof combining such impact data with preprogrammed user biometric data to display risk factor, comprising:
a. a mouthpiece for compatibility with a user's mouth;
b. at least one power source for powering said mouth guard;
c. at least a first accelerometer coupled to said mouthpiece for detecting a linear force;
d. at least a first gyroscope coupled to said mouthpiece for detecting a rotational force; and
e. at least one processing unit coupled to said mouthpiece to receive impact data from said at least a first accelerometer or said at least a first gyroscope, said processing unit capable of transmitting a signal to trigger at least one display indicator; whereas
f. said at least one display indicator coupled to said mouthpiece; said at least one display indicator capable of receiving said signal; and said at least one display indicator for displaying a light.

2. The mouth guard of claim 1 wherein said at least one display indicator capable of displaying at least two distinct color wavelengths of light.

3. The mouth guard of claim 2 wherein said at least one display indicator capable of displaying at least a third distinct color wavelength of light.

4. The mouth guard of claim 1 wherein said at least one display indicator comprises a light source embedded within said mouthpiece.

5. The mouth guard of claim 1 wherein said at least one display indicator comprises a light source adjacent to said mouthpiece.

6. The mouth guard of claim 1
a. wherein said at least one display indicator capable of at least a first display mode; and at least a second display mode distinct from said first display mode;
b. wherein said processing unit capable of calculating when a first predetermined impact threshold is met based on said impact data;
c. said processing unit transmitting a first signal to trigger said at least one display indicator to display the first display mode.
d. wherein said processing unit capable calculating when a second predetermined impact threshold is met based on said impact data;
e. said processing unit transmitting a second signal to trigger said at least one display indicator to display the second display mode.

7. The mouth guard of claim 6 wherein said first display mode is a first color light; and said second display mode is a second color light.

8. The mouth guard of claim 6 wherein said first display mode is a blinking light; and said second display mode is a solid light.

9. The mouth guard of claim 1 further comprising an on-board data input system coupled to said mouthpiece for setting at least one predetermined force threshold.

10. The mouth guard of claim 9 whereby said at on-board data input system receives biometric data to allow said processing unit to set said at least one predetermined threshold.

11. The mouth guard of claim 1 further comprising a switch along said mouth piece for powering on and down said mouth guard.

12. The mouth guard of claim 1 wherein said processing unit comprises memory.

13. The mouth guard of claim 12 wherein said processing unit memory comprises stored biometric data.

14. The mouth guard of claim 12 further comprising at least one ‘on’ mode and at least one ‘down’ mode, wherein said memory persists when mouth guard is ‘down’.

15. The mouth guard of claim 12 wherein said memory persistence is not dependent on said power source.

16. The mouth guard of claim 12 wherein said memory capable of storing past impact data.

17. The mouth guard of claim 16 comprising a time keeping system; said processing unit capable of selectively dismissing impact data temporally

18. The mouth guard of claim 16 comprising a reset function to eliminate impact data from memory.

19. The mouth guard of claim 13 wherein said biometric data comprises a selection of an age category for the user.

20. The mouth guard of claim 13 wherein said biometric data comprises a selection of a weight category for the user.

21. The mouth guard of claim 13 wherein said biometric data comprises a selection of a gender category for the user.

22. The mouth guard of claim 1 wherein said power source comprises a battery coupled to said mouth piece.

23. The mouth guard of claim 22 wherein said battery is rechargeable.

24. The mouth guard of claim 23 wherein said battery is rechargeable via inductive charge.

25. The mouth guard of claim 16
a. Wherein said first predetermined impact threshold indicates a first force incident; and
b. Wherein said second predetermined impact threshold indicates a second force incident.

26. The mouth guard of claim 25 further comprising a time keeping system to selectively eliminate past impact data from memory.

27. The mouth guard of claim 25 further comprising a reset function to eliminate past impact data from memory.

28. The mouth guard of claim 25 wherein said at least one display indicator displays a blinking light when said first predetermined impact threshold is met.

29. The mouth guard of claim 25 wherein said at least one display indicator displays a solid light when said second predetermined impact threshold is met.

30. The mouth guard of claim 6
a. wherein said first predetermined impact threshold indicates a minor force; and
b. wherein said second predetermined impact threshold indicates a major force.

31. The mouth guard of claim 30 wherein said at least one display indicator displays a first color light when said first predetermined impact threshold is met and a second color when said second predetermined impact threshold is met.

32. A method for indicating impact experienced by a user wearing a mouth guard comprising the steps of:
a. providing a mouthpiece to a user, the mouthpiece including means for detecting linear and rotational forces, the mouthpiece including means for displaying;
b. setting at least one predetermined impact threshold for detection by the means for detecting;
c. detecting linear and rotational forces experienced by the mouthpiece;
d. determining when such linear and rotational forces meet the threshold; and
e. displaying a light coupled to the mouthpiece.

33. The method of claim 32 whereby said step of determining comprises a scheme of at least a linear force and at least a rotational force.

34. The method of claim 33 whereby said step of determining comprises using a predictive capability risk function.

35. The method of claim 32 whereby said step of displaying comprises sending a signal to an off-board device for remote display.
